(19) United States
(12) Patent Application Publication
(10) Pub. No.: US 2023/0226468 A1
(43) Pub. Date: Jul. 20, 2023

(54) GAS DETECTING APPARATUSES WITH INTEGRATED FLOW REGULATING COMPONENTS

(71) Applicant: HONEYWELL INTERNATIONAL INC., Charlotte, NC (US)

(72) Inventors: Zuohua LI, Charlotte, NC (US); Yong WANG, Charlotte, NC (US); Tao CHEN, Charlotte, NC (US); Erik JACOBSON, Charlotte, NC (US); Wei YU, Charlotte, NC (US); Li LIU, Charlotte, NC (US)

(21) Appl. No.: 18/148,835

(22) Filed: Dec. 30, 2022

(30) Foreign Application Priority Data
Jan. 17, 2022 (CN) 202210047072.6

(51) Int. Cl. B01D 29/00; B01D 35/02

(52) U.S. Cl. CPC B01D 29/002; B01D 29/0097; B01D 35/02

(57) ABSTRACT

Methods, apparatuses and systems for providing an apparatus such as, for example, a gas detecting apparatus are disclosed herein. An example apparatus may comprise: a manifold defining a gas flow channel that is configured to convey a gaseous substance from a gas inlet on a surface of the manifold; and at least one integrated flow regulating component comprising a filtering element that is configured to filter the gaseous substance, wherein the filtering element is disposed adjacent an orifice that leads to an interior channel of the integrated flow regulating component, and wherein the filtering element is disposed upstream with respect to the orifice.

### CROSS-REFERENCE TO RELATED APPLICATIONS

This application claims priority pursuant to 35 U.S.C. 119(a) to Chinese Application No. 202210047072.6, filed Jan. 17, 2022, which application is incorporated herein by reference in its entirety.

### BACKGROUND

Gas detectors may be utilized to detect and/or measure the concentration level of gaseous substances and/or compounds in a gaseous substance, including, for example, organic compounds and inorganic compounds.

Many gas detectors are plagued with technical challenges and limitations. Through applied effort, ingenuity, and innovation, many of these identified problems have been solved by developing solutions that are included in embodiments of the present disclosure, many examples of which are described in detail herein.

### BRIEF SUMMARY

Various embodiments described herein relate to methods, apparatuses, and systems for providing an apparatus such as, for example, a gas detecting apparatus.

In accordance with various examples of the present disclosure, an apparatus is provided. The apparatus may comprise: a manifold defining a gas flow channel that is configured to convey a gaseous substance from a gas inlet on a surface of the manifold; and at least one integrated flow regulating component comprising a filtering element that is configured to filter the gaseous substance, wherein the filtering element is disposed adjacent an orifice that leads to an interior channel of the integrated flow regulating component, and wherein the filtering element is disposed upstream with respect to the orifice.

In some examples, the filtering element is at least partially contained within a body of the integrated flow regulating component.

In some examples, the filtering element is removably attached to at least a surface of the integrated flow regulating component.

In some examples, the filtering element defines a cavity configured to abut the orifice.

In some examples, the at least one integrated flow regulating component is configured to be removably received within an opening on a surface of the apparatus.

In some examples, the apparatus comprises a gas detecting apparatus.

In some examples, a diameter of the orifice is between 0.2 millimeters (mm) and 2 mm.

In some examples, the filtering element comprises a substantially cylindrical member.

In some examples, the filtering element comprises at least one of a textile or plastic material.

In some examples, the at least one integrated flow regulating component comprises at least one of a metal or plastic material.

In accordance with various examples of the present disclosure, an integrated flow regulating component is provided. The integrated flow regulating component may comprise a filtering element that is configured to filter a gaseous substance, wherein the filtering element is disposed adjacent an orifice that leads to an interior channel of the integrated flow regulating component, and wherein the filtering element is disposed upstream with respect to the orifice.

The foregoing illustrative summary, as well as other exemplary objectives and/or advantages of the disclosure, and the manner in which the same are accomplished, are further explained in the following detailed description and its accompanying drawings.

### BRIEF DESCRIPTION OF THE DRAWINGS

The description of the illustrative embodiments may be read in conjunction with the accompanying figures. It will be appreciated that, for simplicity and clarity of illustration, elements illustrated in the figures have not necessarily been drawn to scale, unless described otherwise. For example, the dimensions of some of the elements may be exaggerated relative to other elements, unless described otherwise. Embodiments incorporating teachings of the present disclosure are shown and described with respect to the figures presented herein, in which:

FIG. 1 illustrates an example conventional apparatus in accordance with various embodiments of the present disclosure;

FIG. 2 illustrates a schematic diagram depicting an example apparatus in accordance with various embodiments of the present disclosure;

FIG. 3 illustrates a side section view of an example portion of an apparatus in accordance with various embodiments of the present disclosure;

FIG. 4 illustrates a schematic diagram depicting an example portion of an apparatus in accordance with various embodiments of the present disclosure;

FIG. 5 illustrates a schematic diagram depicting an example portion of an apparatus in accordance with various embodiments of the present disclosure;

FIG. 6A illustrates a schematic diagram depicting a perspective view of an example flow regulating component in accordance with various embodiments of the present disclosure; and

FIG. 6B illustrates a schematic diagram depicting a perspective view of an example flow regulating component in accordance with various embodiments of the present disclosure.

### DETAILED DESCRIPTION OF THE INVENTION

Some embodiments of the present disclosure will now be described more fully hereinafter with reference to the accompanying drawings, in which some, but not all embodiments of the disclosure are shown. Indeed, these disclosures may be embodied in many different forms and should not be construed as limited to the embodiments set forth herein; rather, these embodiments are provided so that this disclosure will satisfy applicable legal requirements. Like numbers refer to like elements throughout.

The components illustrated in the figures represent components that may or may not be present in various embodiments of the present disclosure described herein such that embodiments may include fewer or more components than those shown in the figures while not departing from the scope of the present disclosure. Some components may be omitted from one or more figures or shown in dashed line for visibility of the underlying components.

The phrases “in an example embodiment,” “some embodiments,” “various embodiments,” and the like generally mean that the particular feature, structure, or characteristic following the phrase may be included in at least one embodiment of the present disclosure, and may be included in more than one embodiment of the present disclosure (importantly, such phrases do not necessarily refer to the same embodiment).

The word “example” or “exemplary” is used herein to mean “serving as an example, instance, or illustration.” Any implementation described herein as “exemplary” is not necessarily to be construed as preferred or advantageous over other implementations.

If the specification states a component or feature “may,” “can,” “could,” “should,” “would,” “preferably,” “possibly,” “typically,” “optionally,” “for example,” “often,” or “might” (or other such language) be included or have a characteristic, that a specific component or feature is not required to be included or to have the characteristic. Such components or features may be optionally included in some embodiments, or may be excluded.

The term “electronically coupled” or “in electronic communication with” in the present disclosure refer to two or more electrical elements (for example, but not limited to, an example processing circuitry, communication module, input/output module memory, humidity sensing component, cooling element, gas detection component) and/or electric circuit(s) being connected through wired means (for example but not limited to, conductive wires or traces) and/or wireless means (for example but not limited to, wireless network, electromagnetic field), such that data and/or information (for example, electronic indications, signals) may be transmitted to and/or received from the electrical elements and/or electric circuit(s) that are electronically coupled.

Various apparatuses (such as, but not limited to, a gas detecting apparatus) may measure a concentration level of volatile organic compounds in a gaseous substance. The term “volatile organic compound” (or “VOC”) refers to organic compounds that may have a high vapor pressure at ordinary room temperature (i.e., they may easily become gases or vapors). Example chemicals in example VOCs may include, for example, but not limited to, formaldehyde, methane, and benzene. A high-level concentration of VOCs in a gaseous substance (for example, indoor air or outdoor air) may cause adverse effect on health and environment. As such, gas detecting apparatuses may be utilized to measure and monitor the concentration level of VOCs in various indoor and/or outdoor locations.

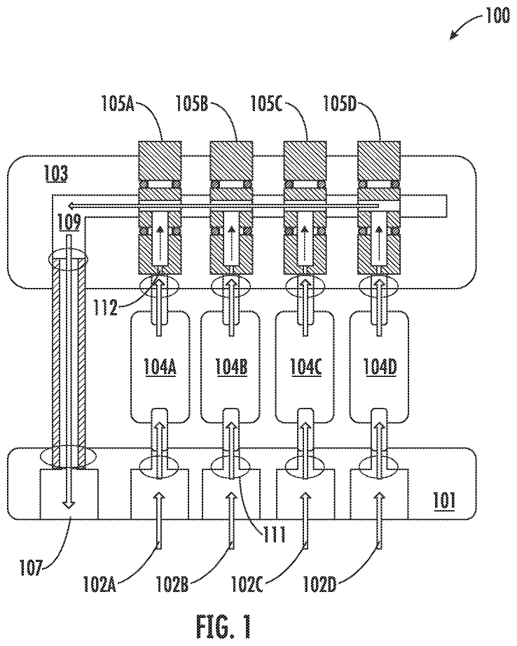

Referring now to FIG. 1, a schematic diagram depicting an example conventional gas detector 100 in accordance with various embodiments of the present disclosure is provided.

In various embodiments, the example gas detector 100 may be at least partially disposed/arranged in a housing. Additionally, the example gas detector 100 is configured to receive a gaseous substance (e.g., air) and convey the gaseous substance from one or more openings/inlets on a surface of the gas detecting apparatus and convey the gaseous substance through one or more gas flow channels to be expelled from the gas detecting apparatus (e.g., caused to exit the gas detecting apparatus) via one or more apertures/openings, or outlets. Thus, the gaseous substance is received through an opening (e.g., first gas inlet 102A) leading to the gas flow channel and conveyed to another opening (e.g., gas outlet 104) where it may be expelled from the gas detector 100.

In particular, as depicted, the example gas detector 100 comprises a first manifold 101, a second manifold 103, a first flow device 105A, a second flow device 105B, a third flow device 105C, a fourth flow device 105D, a first filtering device 104A, a second filtering device 104B, a third filtering device 104C, a fourth filtering device 104D, and defines a gas flow channel 109.

As noted above, the example gas detector 100 comprises a first manifold 101 and a second manifold 103. In various embodiments, each of the first manifold 101 and second manifold 103 defines a plurality of passageways (e.g., gas flow channel 109) through which a sample gaseous substance (e.g., an air sample) may enter, be conveyed, and be expelled from the example gas detector 100. Each of the first manifold 101 and the second manifold 103 may be or comprise a device that is configured to regulate the flow of a gaseous substance (e.g., liquids, gases). Accordingly, the first manifold 101 and the second manifold 103 may be critical for controlling and/or regulating the flow of gaseous substances/air distribution within the example gas detector 100.

As noted above, and as further depicted in FIG. 1, the example gas detector 100 comprises a first flow device 105A, a second flow device 105B, a third flow device 105C, a fourth flow device 105D. Each of the first flow device 105A, the second flow device 105B, the third flow device 105C, and the fourth flow device 105D defines an orifice/opening (e.g., orifice 112) disposed on/defining a first end of the device (i.e., an end portion of the device that is disposed within the second manifold 103) that operates to control/regulate the flow of a gaseous substance (e.g., air sample) through an interior channel of the example device and along an associated flow channel of the gas detector 100.

As further depicted in FIG. 1, the example gas detector 100 comprises a first filtering device 104A, a second filtering device 104B, a third filtering device 104C and a fourth filtering device 104D. As depicted, each of the first filtering device 104A, the second filtering device 104B, the third filtering device 104C, and the fourth filtering device 104D is disposed/arranged between the first manifold 101 and the second manifold 103 and operates to filter out debris or foreign material that may be present in a sample gaseous substance entering an inlet of the gas detector 100. Additionally, each of the first filtering device 104A, the second filtering device 104B, the third filtering device 104C, and the fourth filtering device 104D is connected to and/or positioned adjacent a respective flow device. For example, as shown, the first flow device 105A is disposed/positioned adjacent the first filtering device 104A such that the sample gaseous substance will flow through the first filtering device 104A prior to reaching the first flow device 105A.

As depicted in FIG. 1, a sample gaseous substance (e.g., air sample) may enter the example gas detector 100 via a first gas inlet 102A on a surface of the gas detector 100. The sample gaseous substance (e.g., air sample) may be conveyed via the first manifold 101 along at least a portion of the gas flow channel 109. As further depicted, the sample gaseous substance (e.g., air sample) may flow from the first manifold 101 through the first filtering device 104A and the first flow device 105A that are disposed between the first manifold 101 and the second manifold 103. Additionally, as shown, the first flow device 105A is at least partially disposed within the second manifold 103. As further illustrated, subsequent to flowing through the first flow device 105A/second manifold 103, the sample gaseous substance (e.g., air sample) may exit the gas flow channel 109 of the gas detector 100 via a gas outlet 107 on a surface of the gas detector 100.

As further depicted in FIG. 1, a sample gaseous substance (e.g., air sample) may enter the example gas detector 100 via a second gas inlet 102B on a surface of the gas detector 100. The sample gaseous substance (e.g., air sample) may be conveyed via the first manifold 101 along at least a portion of the gas flow channel 109. In particular, the sample gaseous substance may be conveyed through the second filtering device 104B and the second flow device 105B that are disposed between the first manifold 101 and the second manifold 103. As depicted, the second flow device 105B is at least partially disposed within the second manifold 103. As further illustrated, subsequent to flowing through the second flow device 105B/second manifold 103, the sample gaseous substance (e.g., air sample) may exit the gas flow channel 109 of the gas detector 100 via a gas outlet 107 on a surface of the gas detector 100.

Similarly, as illustrated in FIG. 1, a sample gaseous substance (e.g., air sample) may enter the example gas detector 100 via a third gas inlet 102C on a surface of the gas detector 100. The sample gaseous substance (e.g., air sample) may be conveyed via the first manifold 101 along at least a portion of the gas flow channel 109. In particular, the sample gaseous substance may be conveyed through the third filtering device 104C and the third flow device 105C that are disposed between the first manifold 101 and the second manifold 103. As depicted, the third flow device 105C is at least partially disposed within the second manifold 103. As further illustrated, subsequent to flowing through the third flow device 105C/second manifold 103, the sample gaseous substance (e.g., air sample) may exit the gas flow channel 109 of the gas detector 100 via a gas outlet 107 on a surface of the gas detector 100.

As depicted in FIG. 1, a sample gaseous substance (e.g., air sample) may enter the example gas detector 100 via a fourth gas inlet 102D on a surface of the gas detector 100. The sample gaseous substance (e.g., air sample) may be conveyed via the first manifold 101 along at least a portion of the gas flow channel 109. In particular, the sample gaseous substance may be conveyed through the fourth filtering device 104D and the fourth flow device 105D that are disposed between the first manifold 101 and the second manifold 103. As depicted, the fourth flow device 105D is at least partially disposed within the second manifold 103. As further illustrated, subsequent to flowing through the fourth flow device 105D/second manifold 103, the sample gaseous substance (e.g., air sample) may exit the gas flow channel 109 of the gas detector 100 via a gas outlet 107 on a surface of the gas detector 100.

In some embodiments, the gaseous substance may be conveyed in a direction due to air flow generated by a pump. For example, the pump may generate air flow in the gas flow channel such as by pulling in (e.g., drawing in) a gaseous substance (e.g., input air) through one or more gas inlets (e.g., a first gas inlet 102A) and pushing the gaseous substance (e.g., output air) through one or more gas outlets (e.g., gas outlet 107). In some examples, one or more locations at which the different components of the gas detector 100 make contact with one another may be sealed, as indicated with circles in FIG. 1 (e.g., a first location 111 between the first manifold 101 and the second filtering device 104B).

As noted above, during operations, a sample gaseous substance (e.g., comprising a combination of VOC and non-VOC molecules) may flow through one or more passageways defining a gas flow channel 109 of an example gas detector 100. In some examples, even with the use of filtering components (e.g., first filtering device 104A) an orifice (e.g., orifice 112) of each of the first flow device 105A, the second flow device 105B, the third flow device 105C, and the fourth flow device 105D may become blocked/clogged by material (e.g., particle(s), compounds, and/or debris) that may be present in a sample gaseous substance (e.g., air) as it is conveyed into the gas detector 100 and through the gas flow channel 109. The presence of such unwanted materials may result in reduced sensitivity and accuracy of measurements being generated by the example gas detector.

Moreover, as noted above, in order to properly regulate the flow of a sample gaseous substance into and through the example gas detector, one or more locations (indicated with circles) at which various components are connected and/or make contact with one another (e.g., the first location 111 between the first filtering device 104A and the first gas inlet 102A) may require sealing using, for example, o-rings. Additionally, in various examples, the use of separate filtering devices (e.g., first filtering device 104A) for performing filtering operations in the example gas detector makes the overall structure of the apparatus more complex and difficult/expensive to manufacture/produce. For example, in order to replace or repair a filtering device, the entire gas detecting apparatus may need to be taken apart to allow a technician to reach the filtering device (e.g., first filtering device 104A).

Additionally, as noted above, connections between various devices (e.g., first filtering device 104A and first flow device 105A) may need to be sealed, for example, using o-rings, in order to ensure proper flow of a gaseous substance within the examples gas detecting apparatus. As a result of multiple connection points, the gas detecting apparatus will have a plurality of potential points of failure (e.g., a seal may become damaged/weak, or a connection between two components may become loose) and may be more prone to mechanical failure. Accordingly, poor air flow/sealing in one or more locations of the example gas detector (e.g., first location 111) may also result in inaccurate measurements being generated by the gas detector. As a result of these deficiencies, the sensitivity and performance of the gas detector may be negatively affected. Additionally, having to maintain or repair such gas detectors (e.g., replace one or more filtering devices) may be labor intensive and complex, which increases maintenance costs and reduces operational efficiencies for an end user.

In accordance with various embodiments of the present disclosure, example methods, apparatuses and systems are provided.

For example, the present disclosure may provide an apparatus comprising a manifold. The manifold may define a gas flow channel that is configured to convey a gaseous substance from a gas inlet on a surface of the manifold. The apparatus may comprise at least one integrated flow regulating component comprising a filtering element that is configured to filter the gaseous substance, wherein the filtering element is disposed adjacent an orifice that leads to an interior channel of the integrated flow regulating component, and wherein the filtering element is disposed upstream with respect to the orifice. In some examples, the filtering element is at least partially contained within a body of the integrated flow regulating component. In some examples, the filtering element is removably attached to at least a surface of the integrated flow regulating component. In some examples, the filtering element defines a cavity configured to abut the orifice. In some examples, the at least one integrated flow regulating component is configured to be removably received within an opening on a surface of the apparatus. In some examples, the apparatus comprises a gas detecting apparatus. In some examples, a diameter of the orifice is between 0.2 millimeters (mm) and 2 mm. In some examples, the filtering element comprises a substantially cylindrical member. In some examples, the filtering element comprises at least one of a textile or plastic material. In some examples, the at least one integrated flow regulating component comprises at least one of a metal or plastic material.

In some examples, an integrated flow regulating component is provided. The integrated flow regulating component may comprise a filtering element that is configured to filter a gaseous substance, wherein the filtering element is disposed adjacent an orifice that leads to an interior channel of the integrated flow regulating component, and wherein the filtering element is disposed upstream with respect to the orifice. In some examples, the filtering element is at least partially contained within a body of the integrated flow regulating component. In some examples, the filtering element is removably attached to at least a surface of the integrated flow regulating component. In some examples, the filtering element defines a cavity configured to abut the orifice. In some examples, the integrated flow regulating component is configured to be removably received within an opening on a surface of an apparatus. In some examples, the apparatus comprises a gas detecting apparatus. In some examples, a diameter of the orifice is between 0.2 mm and 2 mm. In some examples, the filtering element comprises a substantially cylindrical member. In some examples, the filtering element comprises at least one of a textile or plastic material. In some examples, the integrated flow regulating component comprises at least one of a metal or plastic material.

In some embodiments, utilizing the techniques disclosed herein, a noticeable reduction in pressure drop may be observed in an example apparatus/system (e.g., gas detecting apparatus), which may improve overall accuracy and performance of the example apparatus/system.

While some of the embodiments herein provide an example gas detecting apparatus, it is noted that the scope of the present disclosure is not limited to such embodiments. For example, in some examples, an example gas detection component in accordance with the present disclosure may be in other forms.

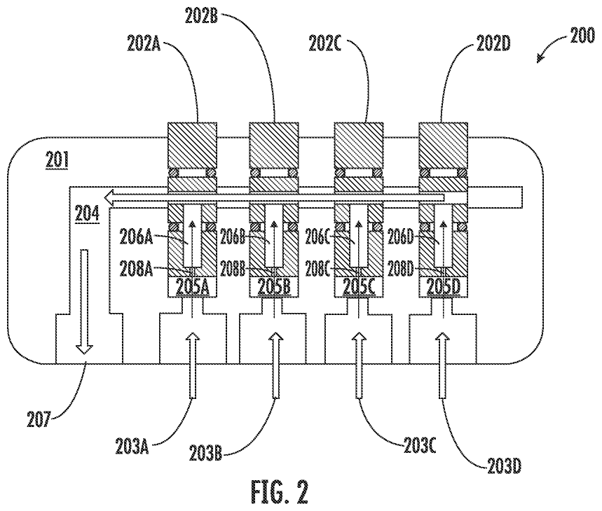

Referring now to FIG. 2, a schematic diagram depicting an example apparatus 200 in accordance with various embodiments of the present disclosure is provided. In various embodiments, the apparatus 200 may be or comprise at least a portion of a gas detecting apparatus, a hydraulic system, air/fluid delivery system, or the like. In some embodiments, the apparatus 200 may be at least partially disposed/arranged within a housing. Accordingly, the apparatus 200 may be configured to control and/or regulate the flow of gaseous substances and/or air distribution.

As illustrated in FIG. 2, the example apparatus 200 (e.g., gas detecting apparatus) defines a gas flow channel 204. The gas flow channel 204 may refer to a passageway or plurality of channels beginning with at least one gas inlet (e.g., as depicted, first gas inlet 203A) and terminating with at least one gas outlet (e.g., as depicted, gas outlet 207) through which a sample gaseous substance (e.g., an air sample) may enter, be conveyed and be expelled from the example apparatus 200 (e.g., gas detecting apparatus). The gas flow channel 204 may be or comprise, for example, without limitation, a pipe, conduit, tubular structure and/or the like. In particular, the apparatus 200 (e.g., gas detecting apparatus) comprises a manifold 201 (e.g., a single manifold) comprising a plurality of integrated flow regulating components. The terms “integrated flow regulating component” and “flow regulating component” are used interchangeably herein. In particular, the apparatus 200 comprises a first flow regulating component 202A, a second flow regulating component 202B, a third flow regulating component 202C, and a fourth flow regulating component 202D.

Referring again to FIG. 2, a gaseous substance (for example, an air sample) may enter the apparatus 200 through a first gas inlet 203A, a second gas inlet 203B, a third gas inlet 203C and a fourth gas inlet 203D. Accordingly, each of the first gas inlet 203A, the second gas inlet 203B, the third gas inlet 203C, and the fourth gas inlet 203D may refer to an opening on a surface of the housing of the apparatus 200 that is configured to receive a sample gaseous substance (e.g., air sample). In some embodiments, the gas outlet 207 may refer to an opening on a surface of the housing of the apparatus 200 that may be distinct from the openings on the surface associated with the first gas inlet 203A, the second gas inlet 203B, the third gas inlet 203C, and the fourth gas inlet 203D. In some embodiments, the gaseous substance may be conveyed in a direction due to air flow generated by a pump (for example, without limitation a compressor, a vacuum pump, a manual pump, a motorized pump or the like). For example, the pump may generate air flow in the gas flow channel 204 such as by pulling in (e.g., drawing in) a gaseous substance (e.g., input air) through one or more gas inlets (e.g., the first gas inlet 203A, the second gas inlet 203B, the third gas inlet 203C, and the fourth gas inlet 203D) and pushing the gaseous substance (e.g., output air) through the gas outlet 207.

As further depicted in FIG. 2, the example apparatus 200 comprises a manifold 201 which defines a plurality of passageways/channels (e.g., a gas flow channel 204) through which a sample gaseous substance (e.g., an air sample) may enter, be conveyed, and be expelled from the example apparatus 200. In various examples, the example manifold 201 is configured to regulate the flow of a gaseous substance (e.g., liquids, gases) into and through the apparatus 200. The example manifold 201 may be or comprise metal, plastic, combinations thereof, and/or the like.

As noted above, and as illustrated in FIG. 2, the example apparatus 200 comprises a first flow regulating component 202A, a second flow regulating component 202B, a third flow regulating component 202C, and a fourth flow regulating component 202D. In various examples, each flow regulating component 202A, 202B, 202C and 202D defines an orifice/opening (e.g., first orifice 208A) that operates to control/regulate the flow of a gaseous substance (e.g., air sample) along an interior passageway/flow channel (e.g., interior channel 206A) and along an associated passageway/gas flow channel 204 of the apparatus 200.

As depicted, each flow regulating component 202A, 202B, 202C and 202D may be or comprise a substantially cylindrical member or body defining an interior channel (e.g., interior channel 206A). Additionally, each flow regulating component 202A, 202B, 202C and 202D is configured to be removably inserted/engaged within an aperture on a surface of the example apparatus 200. In some embodiments, each flow regulating component 202A, 202B, 202C and 202D may comprise metal, plastic, combinations thereof, and/or the like. In some embodiments, as shown, at least a portion of the interior passageway/flow channel (e.g., interior channel 206A) of the flow regulating component (e.g., first flow regulating component 202A) is configured to form, define, and/or lead to at least a portion of a main passageway/flow channel (e.g., gas flow channel 204) through the manifold 201 of the example apparatus 200. For example, as depicted, an internal vertically oriented channel of the first flow regulating component 202A is configured to intersect with a horizontally oriented portion of the gas flow channel 204 so that a sample gaseous substance can flow into the first flow regulating component 202A and into the gas flow channel 204.

Additionally, each flow regulating component 202A, 202B, 202C and 202D is also configured to filter out materials (e.g., debris, particles, or the like) that may be present in the sample gaseous substance entering an inlet of the apparatus 200.

By way of example, as depicted in FIG. 2, the first flow regulating component 202A defines an orifice 207A that is disposed adjacent a first filtering element 205A. As shown, the first filtering element 205A is positioned upstream with respect to the orifice leading to an interior channel 206A of the first flow regulating component 202A. The term upstream may refer to a location of a first component in a gas flow channel with respect to second component in the gas flow channel, based at least in part on the direction of flow of a gaseous substance within the gas flow channel. For example, if a gaseous substance flows to component A, and subsequently flows to component B, then component A is upstream with respect to component B. Similarly, the term downstream may refer to a location of a first component in a gas flow channel with respect to second component in the gas flow channel, based at least in part on the direction of flow of a gaseous substance within the gas flow channel. For example, if a gaseous substance flows to component A, and subsequently flows to component B, then component B is downstream with respect to component A. In some examples, the example orifice may define an opening with a diameter between 0.2 millimeters (mm) and 2 mm. In other words, each flow regulating component 202A, 202B, 202C and 202D comprises a filtering element (e.g., first filtering element 205A), an orifice (e.g., orifice 208A) and an interior flow channel (e.g., interior channel 206A) configured such that the filtering element is integrated with/into the main body of the flow regulating component (e.g., first flow regulating component 202A). In some examples, the filtering element (e.g., first filtering element 205A) is removably attached and/or coupled to the main body of the first flow regulating component 202A. Accordingly, the example first flow regulating component 202A may define a unitary body when in use/positioned within the example manifold 201. In some examples, the filtering element (e.g., first filtering element 205A) may be or comprise biodegradable materials, textile(s), polystyrene, polyvinyl chloride, polyethylene, combinations thereof, and/or the like. In some examples, the filtering element (e.g., first filtering element 205A) may be or comprise a molded part.

As depicted in FIG. 2, a sample gaseous substance (e.g., air sample) may enter the apparatus 200 via the first gas inlet 203A on a surface of the apparatus 200. The sample gaseous substance (e.g., air sample) may be conveyed through the manifold 201, along at least a portion of the gas flow channel 204 via the first flow regulating component 202A. As shown, and as noted above, the first flow regulating component 202A is at least partially disposed within the manifold 201. As further depicted, the sample gaseous substance (e.g., air sample) may flow through a first filtering element 205A of the first flow regulating component 202A. Additionally, as depicted, at least a portion of the first flow regulating component 202A (e.g., an end portion) protrudes from a surface of the manifold 201 in order to facilitate removal for repair or replacement. In some examples, as shown, the first filtering element 205A is located upstream with respect to an orifice 208A of the first flow regulating component 202A. As further illustrated, subsequent to flowing through the first flow regulating component 202A, the sample gaseous substance (e.g., air sample) may exit the gas flow channel 204 of the apparatus 200 via a gas outlet 207 on a surface of the apparatus 200.

As illustrated in FIG. 2, a sample gaseous substance (e.g., air sample) may enter the apparatus 200 via the second gas inlet 203B on a surface of the apparatus 200. The sample gaseous substance (e.g., air sample) may be conveyed through the manifold 201, along at least a portion of the gas flow channel 204 via the second flow regulating component 202B. As depicted, the second flow regulating component 202B is at least partially disposed within the manifold 201. As further depicted, the sample gaseous substance (e.g., air sample) may flow through a second filtering element 205B of the second flow regulating component 202B. Additionally, as depicted, at least a portion of the second flow regulating component 202B (e.g., an end portion) protrudes from a surface of the manifold 201 in order to facilitate removal for repair or replacement. In some examples, as shown, the second filtering element 205B is located upstream with respect to an orifice 208B of the second flow regulating component 202B. As further illustrated, subsequent to flowing through the second flow regulating component 202B, the sample gaseous substance (e.g., air sample) may exit the gas flow channel 204 of the apparatus 200 via a gas outlet 207 on a surface of the apparatus 200.

As illustrated in FIG. 2, a sample gaseous substance (e.g., air sample) may enter the apparatus 200 via the third gas inlet 203C on a surface of the apparatus 200. The sample gaseous substance (e.g., air sample) may be conveyed through the manifold 201, along at least a portion of the gas flow channel 204 via the third flow regulating component 202C. As depicted, the third flow regulating component 202C is at least partially disposed within the manifold 201. As further depicted, the sample gaseous substance (e.g., air sample) may flow through a third filtering element 205C of the third flow regulating component 202C. Additionally, as depicted, at least a portion of the third flow regulating component 202C (e.g., an end portion) protrudes from a surface of the manifold 201 in order to facilitate removal for repair or replacement. In some examples, as shown, the third filtering element 205C is located upstream with respect to an orifice 208B of the third flow regulating component 202C. As further illustrated, subsequent to flowing through the third flow regulating component 202C, the sample gaseous substance (e.g., air sample) may exit the gas flow channel 204 of the apparatus 200 via a gas outlet 207 on a surface of the apparatus 200.

As further depicted in FIG. 2, a sample gaseous substance (e.g., air sample) may enter the apparatus 200 via the fourth gas inlet 203D on a surface of the apparatus 200. The sample gaseous substance (e.g., air sample) may be conveyed through the manifold 201, along at least a portion of the gas flow channel 204 via the fourth flow regulating component 202D. As depicted, the fourth flow regulating component 202D is at least partially disposed within the manifold 201. As further depicted, the sample gaseous substance (e.g., air sample) may flow through a fourth filtering element 205D of the fourth flow regulating component 202D. Additionally, as depicted, at least a portion of the fourth flow regulating component 202D (e.g., an end portion) protrudes from a surface of the manifold 201 in order to facilitate removal for repair or replacement. In some examples, as shown, the fourth filtering element 205D is located upstream with respect to an orifice 208D of the fourth flow regulating component 202D. As further illustrated, subsequent to flowing through the fourth flow regulating component 202D, the sample gaseous substance (e.g., air sample) may exit the gas flow channel 204 of the apparatus 200 via a gas outlet 207 on a surface of the apparatus 200.

While FIG. 2 provides an example apparatus 200, it is noted that the scope of the present disclosure is not limited to the example shown in FIG. 2. In some examples, an apparatus in accordance with the present disclosure may comprise one or more additional and/or alternative elements, and/or may be structured/positioned differently than those illustrated in FIG. 2.

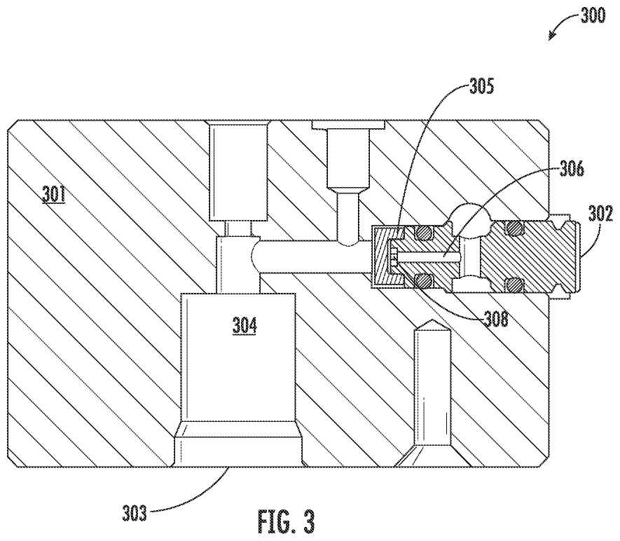

Referring now to FIG. 3, a schematic diagram depicting a side section view of at least a portion of an example apparatus 300 in accordance with various embodiments of the present disclosure is provided. The apparatus 300 may be similar or identical to the apparatus 200 described above in connection with FIG. 2.

In various embodiments, the apparatus 300 may be or comprise at least a portion of a gas detecting apparatus, a hydraulic system, air/fluid delivery system, or the like. In some embodiments, the apparatus 300 may be at least partially disposed/arranged within a housing. Accordingly, the apparatus 300 may be configured to control and/or regulate the flow of gaseous substances and/or air distribution.

As illustrated in FIG. 3, the example apparatus 300 (e.g., gas detecting apparatus) defines a gas flow channel 304. The gas flow channel 304 may refer to a passageway or plurality of channels beginning with at least one gas inlet (e.g., as depicted, gas inlet 303) and terminating with at least one gas outlet through which a sample gaseous substance (e.g., an air sample) may enter, be conveyed and be expelled from the example apparatus 300 (e.g., gas detecting apparatus). The gas flow channel 304 may be or comprise, for example, without limitation, a pipe, conduit, tubular structure and/or the like. In particular, the apparatus 300 (e.g., gas detecting apparatus) comprises a manifold 301 (e.g., a single manifold) comprising at least one integrated flow regulating component 302.

Referring again to FIG. 3, a gaseous substance (for example, an air sample) may enter the apparatus 300 through the gas inlet 303. The gas inlet 303 may be an opening on a surface of the housing of the apparatus 300 that is configured to receive a sample gaseous substance (e.g., air sample). In some embodiments, the gaseous substance may be conveyed in a direction due to air flow generated by a pump (for example, without limitation a compressor, a vacuum pump, a manual pump, a motorized pump or the like). For example, the pump may generate air flow in the gas flow channel 304 such as by pulling in (e.g., drawing in) a gaseous substance (e.g., input air) through one or more gas inlets (e.g., gas inlet 303) and pushing the gaseous substance (e.g., output air) through a gas outlet.

As further depicted in FIG. 3, the example apparatus 300 comprises a manifold 301 which defines a plurality of passageways/channels (e.g., a gas flow channel 304) through which a sample gaseous substance (e.g., an air sample) may enter, be conveyed and be expelled from the example apparatus 300. In various examples, the example manifold 301 is configured to regulate the flow of a gaseous substance (e.g., liquids, gases) into and through the apparatus 300. The example manifold 301 may be or comprise metal, plastic, combinations thereof, and/or the like.

In various examples, the flow regulating component 302 defines an orifice 308 that operates to control/regulate the flow of a gaseous substance (e.g., air sample) along an interior channel 306 of the flow regulating component 302 and along an associated passageway/gas flow channel 304 of the apparatus 300. As depicted, the flow regulating component 302 comprises a substantially cylindrical member or body defining at least one interior channel (e.g., interior channel 306). Additionally, the flow regulating component 302 is configured to be removably inserted/engaged within an aperture on a surface of the example apparatus 300. In some embodiments, the flow regulating component may comprise metal, plastic, combinations thereof, and/or the like. In some embodiments, as shown, at least a portion of the interior passageway/flow channel (e.g., interior channel 306) of the flow regulating component 302 forms, defines, and/or leads to at least a portion of a main passageway/flow channel (e.g., gas flow channel 304) through the manifold 301 of the example apparatus 300.

Additionally, as depicted in FIG. 3, the flow regulating component 302 is configured to filter out materials (e.g., debris, particles, or the like) that may be present in the sample gaseous substance entering an inlet of the apparatus 300. By way of example, as depicted in FIG. 3, the flow regulating component 302 defines an orifice 308 that is disposed adjacent a filtering element 305 which defines an first end/outer surface of the flow regulating component 302. As further depicted, the filtering element 305 defines a cavity that is configured to abut the orifice 308 of the flow regulating component 302. In some examples, the filtering element 305 may be at least partially arranged, contained and/or housed within the body of the flow regulating component 302. Thus, in various embodiments, the filtering element 305 may be integrated into/with the flow regulating component 302 (to define a unitary body). In some examples, the filtering element 305 is removably attached and/or coupled to the main body of the first flow regulating component 305. As shown, the filtering element 305 is positioned upstream with respect to the orifice 308 leading to an interior channel 306 of the flow regulating component 302. In some examples, the example orifice 308 may define an opening with a diameter between 0.2 millimeters (mm) and 2 mm (in some examples, 6 mm).

As depicted in FIG. 3, a sample gaseous substance (e.g., air sample) may enter the apparatus 300 via the gas inlet 303 on a surface of the apparatus 300. The sample gaseous substance (e.g., air sample) may be conveyed through the manifold 301 and reach the flow regulating component 302. As shown, and as noted above, the flow regulating component is at least partially disposed within the manifold 301. As further depicted, the sample gaseous substance (e.g., air sample) may flow through the filtering element 305 of the flow regulating component 302 and subsequently flow through the orifice 308. Additionally, as depicted, at least a portion of the flow regulating component 302 (e.g., a second end/outer surface, opposite the first end) protrudes from a surface of the manifold 301 in order to facilitate removal for repair or replacement. Accordingly, repair or replacement of a filtering element 305 can be completed without taking apart the apparatus 300.

While FIG. 3 provides an example apparatus 300, it is noted that the scope of the present disclosure is not limited to the example shown in FIG. 3. In some examples, an apparatus in accordance with the present disclosure may comprise one or more additional and/or alternative elements, and/or may be structured/positioned differently than those illustrated in FIG. 3.

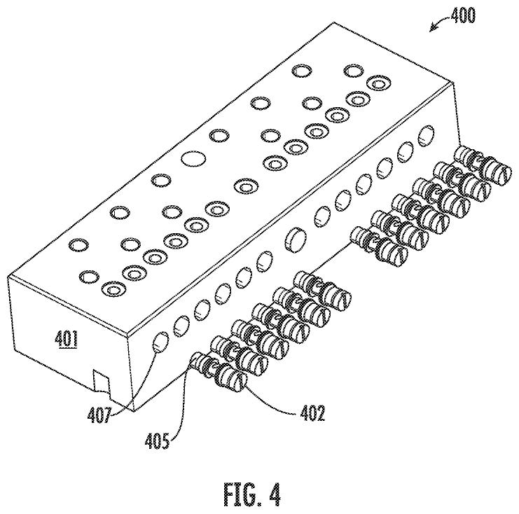

Referring now to FIG. 4, a schematic diagram depicting a side perspective view of at least a portion of an example apparatus 400 in accordance with various embodiments of the present disclosure is provided. The apparatus 400 may be similar or identical to the apparatus 300 described above in connection with FIG. 3.

In various embodiments, the apparatus 400 may be or comprise at least a portion of a gas detecting apparatus, a hydraulic system, air/fluid delivery system, or the like. In some embodiments, the apparatus 400 may be at least partially disposed/arranged within a housing. In various examples, the apparatus 400 may be configured to control and/or regulate the flow of gaseous substances and/or air distribution. In particular, as depicted, the example apparatus 400 comprises a manifold 401 and comprises a plurality of integrated flow regulating components (e.g., flow regulating component 402).

As depicted in FIG. 4, the example apparatus 400 comprises a manifold 401 which defines a plurality of passageways/channels through which a sample gaseous substance (e.g., an air sample) may enter, be conveyed and be expelled from the example apparatus 400. In various examples, the example manifold 401 is configured to regulate the flow of a gaseous substance (e.g., liquids, gases) into and through the apparatus 400. The example manifold 401 may be or comprise metal, plastic, combinations thereof, and/or the like.

As noted above, and as further depicted in FIG. 4, the example apparatus 400 comprises a plurality of integrated flow regulating components (e.g., flow regulating component 402). Each of the plurality of integrated flow regulating components may be similar or identical to flow regulating component 302 discussed above in connection with FIG. 3. For example, each integrated flow regulating component (e.g., integrated flow regulating component 402) may define an orifice that operates to control/regulate the flow of a gaseous substance (e.g., air sample) along an interior channel of the integrated flow regulating component (e.g., integrated flow regulating component 402) and along an associated passageway/gas flow channel of the apparatus 400. Additionally, as depicted, each integrated flow regulating component (e.g., integrated flow regulating component 402) may comprise an integrated filtering element. For example, as shown integrated flow regulating component 402 comprises a filtering element 405.

As depicted, each of the plurality of integrated flow regulating components (e.g., integrated flow regulating component 402) comprises a substantially cylindrical member or body defining at least one interior channel. In some embodiments, each integrated flow regulating component may be or comprise metal, plastic, combinations thereof, and/or the like. In some embodiments, at least a portion of the interior passageway/flow channel of each integrated flow regulating component (e.g., integrated flow regulating component 402) forms, defines, and/or leads to at least a portion of a main passageway/flow channel through the manifold 401 of the example apparatus 400.

Additionally, as depicted, each of the plurality of integrated flow regulating components is configured to be removably inserted/engaged within an aperture on a surface of the example apparatus 400. For example, as illustrated, the integrated flow regulating component 402 is configured to be removably inserted/engaged within an aperture 407 on a side surface of the example apparatus 400.

As depicted in FIG. 4, the filtering element 405 of the integrated flow regulating component 402 is configured to filter out materials (e.g., debris, particles, or the like) that may be present in the sample gaseous substance entering an inlet of the apparatus 400.

In various embodiments, a sample gaseous substance (e.g., air sample) may enter the apparatus 400 via a gas inlet on a surface of the apparatus 400. The sample gaseous substance (e.g., air sample) may be conveyed through the manifold 401 and reach the flow regulating component 402. As shown, and as noted above, the flow regulating component 402 is at least partially disposed within the manifold 401. The sample gaseous substance (e.g., air sample) may flow through the filtering element 405 of the flow regulating component 402 (and subsequently flow through an orifice). Additionally, as depicted, at least a portion of the flow regulating component 402 (e.g., an outer surface) is configured to protrude from a surface (e.g., side surface of the manifold 401) of the apparatus 400 in order to facilitate removal for repair or replacement. Accordingly, repair or replacement of a filtering element 405 can be completed without taking apart the apparatus 400.

While FIG. 4 provides an example apparatus 400, it is noted that the scope of the present disclosure is not limited to the example shown in FIG. 4. In some examples, an apparatus in accordance with the present disclosure may comprise one or more additional and/or alternative elements, and/or may be structured/positioned differently than those illustrated in FIG. 4.

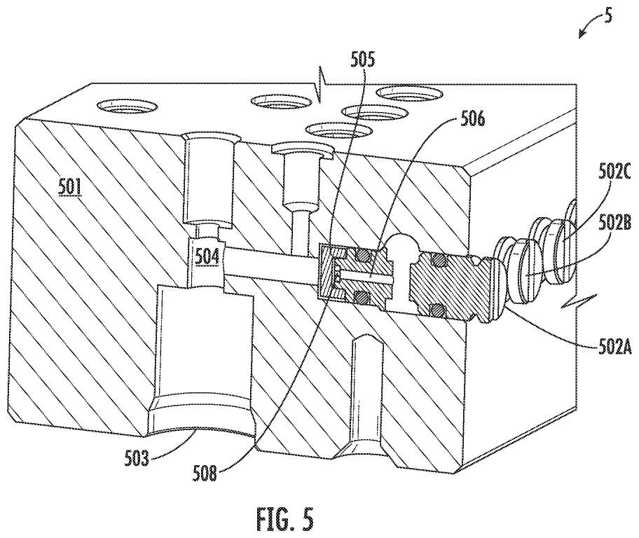

Referring now to FIG. 5, a schematic diagram depicting a side section view of at least a portion of an example apparatus 500 in accordance with various embodiments of the present disclosure is provided. The apparatus 500 may be similar or identical to the apparatus 400 described above in connection with FIG. 4.

Similarly, the apparatus 500 may be or comprise at least a portion of a gas detecting apparatus, a hydraulic system, air/fluid delivery system, or the like. In some embodiments, the apparatus 500 may be at least partially disposed/arranged within a housing. Accordingly, the apparatus 500 may be configured to controlling and/or regulating the flow of gaseous substances and/or air distribution.

As illustrated in FIG. 5, the example apparatus 500 (e.g., gas detecting apparatus) defines a gas flow channel 504. The gas flow channel 504 may refer to a passageway or plurality of channels beginning with at least one gas inlet (e.g., as depicted, gas inlet 503) and terminating with at least one gas outlet through which a sample gaseous substance (e.g., an air sample) may enter, be conveyed and be expelled from the example apparatus 500 (e.g., gas detecting apparatus). The gas flow channel 504 may be or comprise, for example, without limitation, a pipe, conduit, tubular structure and/or the like. In particular, the apparatus 500 (e.g., gas detecting apparatus) comprises a manifold 501 (e.g., a single manifold) comprising at least one integrated flow regulating component (e.g., as depicted, at least a first integrated flow regulating component 502A, a second integrated flow regulating component 502B, and third integrated flow regulating component 502C).

Referring again to FIG. 5, a gaseous substance (for example, an air sample) may enter the apparatus 500 through the gas inlet 503. The gas inlet 503 may be an opening on a surface of the housing of the apparatus 500 that is configured to receive a sample gaseous substance (e.g., air sample). In some embodiments, the gaseous substance may be conveyed in a direction due to air flow generated by a pump (for example, without limitation a compressor, a vacuum pump, a manual pump, a motorized pump or the like). For example, the pump may generate air flow in the gas flow channel 504 such as by pulling in (e.g., drawing in) a gaseous substance (e.g., input air) through one or more gas inlets (e.g., gas inlet 503) and pushing the gaseous substance (e.g., output air) through a gas outlet.

As further depicted in FIG. 5, the example apparatus 500 comprises a manifold 501 which defines a plurality of passageways/channels (e.g., a gas flow channel 504) through which a sample gaseous substance (e.g., an air sample) may enter, be conveyed and be expelled from the example apparatus 500. In various examples, the example manifold 501 is configured to regulate the flow of a gaseous substance (e.g., liquids, gases) into and through the apparatus 500. The example manifold 501 may be or comprise metal, plastic, combinations thereof, and/or the like.

As noted above, the example apparatus comprises at least an integrated flow regulating component (e.g., flow regulating component 502A). The flow regulating component 502A defines an orifice 508 that operates to control/regulate the flow of a gaseous substance (e.g., air sample) along an interior channel 506 of the flow regulating component 502A and along an associated passageway/gas flow channel 504 of the apparatus 500. As depicted, the flow regulating component 502A comprises a substantially cylindrical member or body defining at least one interior channel (e.g., interior channel 306). Additionally, the flow regulating component 502A is configured to be removably inserted/engaged within an aperture on a surface (e.g., as depicted, a side surface) of the example apparatus 500. In some embodiments, the flow regulating component 502A may comprise metal, plastic, combinations thereof, and/or the like. In some embodiments, as shown, at least a portion of the interior passageway/flow channel (e.g., interior channel 306) of the flow regulating component 502A forms, defines, and/or leads to at least a portion of a main passageway/flow channel (e.g., gas flow channel 504) through the manifold 501 of the example apparatus 500.

As further depicted in FIG. 5, the flow regulating component 502A comprises a filtering element 505 that operates to filter out materials (e.g., debris, particles, or the like) that may be present in the sample gaseous substance entering an inlet of the apparatus 500. By way of example, as depicted in FIG. 5, the flow regulating component 502A defines an orifice 308 that is disposed adjacent a filtering element 505 which defines an first end/outer surface of the flow regulating component 502A. In some examples, the filtering element 505 may be at least partially arranged, contained and/or housed within the body of the flow regulating component 502A. Thus, in various embodiments, the filtering element 505 may be integrated into/with the flow regulating component 502A (to define a unitary body). In some examples, the filtering element 505 is removably attached and/or coupled to the main body of the first flow regulating component 502A. As shown, the filtering element 505 is positioned upstream with respect to the orifice 508 leading to an interior channel 506 of the flow regulating component 502A. In some examples, the orifice 308 may define an opening with a diameter between 0.2 millimeters (mm) and 2 mm (in some examples, 6 mm).

As illustrated in FIG. 5, a sample gaseous substance (e.g., air sample) may enter the apparatus 500 via the gas inlet 503 on a surface of the apparatus 500. The sample gaseous substance (e.g., air sample) may be conveyed through the manifold 501 and reach the flow regulating component 502A. As shown, and as noted above, the flow regulating component is at least partially disposed within the manifold 501. As further depicted, the sample gaseous substance (e.g., air sample) may flow through the filtering element 505 of the flow regulating component 502A and subsequently flow through the orifice 508. Additionally, as depicted, at least a portion of the flow regulating component 502A (e.g., a second end/outer surface, opposite the first end) protrudes from a surface of the manifold 501 in order to facilitate removal for repair or replacement, such that repair/replacement of a filtering element 505 can be completed without taking apart the apparatus 500.

While FIG. 5 provides an example apparatus 500, it is noted that the scope of the present disclosure is not limited to the example shown in FIG. 5. In some examples, an apparatus in accordance with the present disclosure may comprise one or more additional and/or alternative elements, and/or may be structured/positioned differently than those illustrated in FIG. 5.

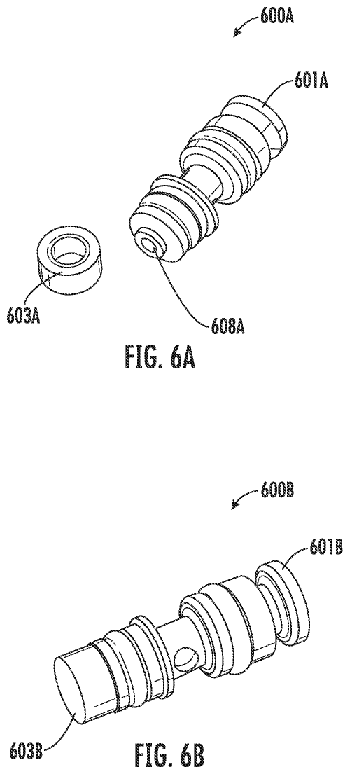

Referring now to FIG. 6A and FIG. 6B, schematic diagrams depicting views of an example flow regulating component 600A and 600B in accordance with various embodiments of the present disclosure are provided.

As depicted, the flow regulating component 600A comprises a substantially cylindrical member or body. In various examples, as depicted, an outer surface of the example flow regulating component 600A may define/comprise a plurality of grooves. Additionally, in various examples, at least a portion of the flow regulating component 600A is configured to be removably inserted/engaged within an aperture on a surface of an example apparatus. In some embodiments, the flow regulating component 600A may comprise metal, plastic, combinations thereof, and/or the like. In some embodiments, as shown, at least a portion of the interior passageway/flow channel of the flow regulating component 600A forms, defines, and/or leads to at least a portion of a main passageway/flow channel (e.g., through a manifold of the example apparatus). In various examples, the flow regulating component 600A defines an orifice 608A that operates to control/regulate the flow of a gaseous substance (e.g., air sample) along an interior channel of the flow regulating component 600A and along an associated passageway/gas flow channel of an apparatus. Additionally, as depicted in FIG. 4, the flow regulating component 600A comprises a filtering element 603A that is configured to filter out materials (e.g., debris, particles, or the like) that may be present in the sample gaseous substance entering an inlet of the example apparatus By way of example, as depicted in FIG. 4, the orifice 608A of the flow regulating component 600A is configured to be positioned adjacent the filtering element 603A to form a first end/outer surface of the flow regulating component 600A. As further depicted, the filtering element 603A defines a cavity that is configured to abut the orifice 608A of the flow regulating component 600A. In some examples, the filtering element 603A may be or comprise a removeable endcap of the flow regulating component 600A. As shown, the filtering element 603A is positioned upstream with respect to the orifice 608 leading to an interior channel of the flow regulating component 600A. In some examples, the example orifice 608A may define an opening with a diameter between 0.2 millimeters (mm) and 2 mm. In some examples, as depicted, the filtering element 603A is configured to be removably attached to at least a surface of the body of the flow regulating component 600A (forming an integrated component/device).

Referring now to FIG. 6B, a schematic diagram depicting another example view of the flow regulating component 600B in accordance with various embodiments of the present disclosure is provided. The flow regulating component 600B may be similar and/or identical to the flow regulating component 600A discussed above in connection with FIG. 6A. As depicted, the flow regulating component 600B comprises a substantially cylindrical member or body. At least a portion of the flow regulating component 600B may be configured to be removably inserted/engaged within an aperture on a surface of an example apparatus. In some embodiments, the flow regulating component 600B may comprise metal, plastic, combinations thereof, and/or the like. In some embodiments, as shown, at least a portion of the interior passageway/flow channel of the flow regulating component 600 forms, defines, and/or leads to at least a portion of a main passageway/flow channel (e.g., through a manifold of the example apparatus). In various examples, the flow regulating component 600B defines an orifice that operates to control/regulate the flow of a gaseous substance (e.g., air sample) along an interior channel of the flow regulating component 600B and along an associated passageway/gas flow channel of an apparatus. As further depicted, the flow regulating component 600B comprises a filtering element 603B that is configured to filter out materials (e.g., debris, particles, or the like) that may be present in the sample gaseous substance entering an inlet of the example apparatus By way of example, as depicted in FIG. 6B, the filtering element 603B may define a first end/outer surface of the flow regulating component 600B. The filtering element 603B may be positioned upstream with respect to an orifice leading to an interior channel of the flow regulating component 600B. In some examples, as depicted, the filtering element 603B may be removably attached to at least a surface of the body of the flow regulating component 600B (forming an integrated component/device). In various examples, the flow regulating components described herein have a simplified structure and reduced size/packaging. Accordingly, these flow regulating components are less complex and cheaper to produce, as well as easier to maintain, than conventional devices. Additionally, unlike conventional devices, the example flow regulating components and associated apparatuses will have very few potential mechanical points of failure and will not require sealing in a plurality of locations. Thus, the responsiveness of the apparatus is not adversely affected and optimal flow of a gaseous substance (e.g., air sample) therethrough, and accuracy of measurements can be maintained.

As described above and as will be appreciated based on this disclosure, embodiments of the present disclosure may be configured as systems, apparatuses, methods, mobile devices, backend network devices, computer program products, other suitable devices, and combinations thereof. Accordingly, embodiments may comprise various means including entirely of hardware or any combination of software with hardware. Furthermore, embodiments may take the form of a computer program product on at least one non-transitory computer-readable storage medium having computer-readable program instructions (e.g., computer software) embodied in the storage medium. Any suitable computer-readable storage medium may be utilized including non-transitory hard disks, CD-ROMs, flash memory, optical storage devices, or magnetic storage devices. As will be appreciated, any computer program instructions and/or other type of code described herein may be loaded onto a computer, processor or other programmable apparatus' circuitry to produce a machine, such that the computer, processor, or other programmable circuitry that executes the code on the machine creates the means for implementing various functions, including those described herein. In some embodiments, features of the present disclosure may comprise, or be communicatively coupled to, an application specific integrated circuit (ASIC).

While various embodiments in accordance with the principles disclosed herein have been shown and described above, modifications thereof may be made by one skilled in the art without departing from the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above, but is defined by the claims which follow, that scope including all equivalents of the subject matter of the claims. Each and every claim is incorporated as further disclosure into the specification and the claims are embodiment(s) of the present disclosure. Furthermore, any advantages and features described above may relate to specific embodiments, but shall not limit the application of such issued claims to processes and structures accomplishing any or all of the above advantages or having any or all of the above features.

In addition, the section headings used herein are provided for consistency with the suggestions under 37 C.F.R. § 1.77 or to otherwise provide organizational cues. These headings shall not limit or characterize the disclosure set out in any claims that may issue from this disclosure. For instance, a description of a technology in the “Background” is not to be construed as an admission that certain technology is prior art to any disclosure in this disclosure. Neither is the “Summary” to be considered as a limiting characterization of the disclosure set forth in issued claims. Furthermore, any reference in this disclosure to “disclosure” or “embodiment” in the singular should not be used to argue that there is only a single point of novelty in this disclosure. Multiple embodiments of the present disclosure may be set forth according to the limitations of the multiple claims issuing from this disclosure, and such claims accordingly define the disclosure, and their equivalents, that are protected thereby. In all instances, the scope of the claims shall be considered on their own merits in light of this disclosure, but should not be constrained by the headings set forth herein.

Also, systems, subsystems, apparatuses, techniques, and methods described and illustrated in the various embodiments as discrete or separate may be combined or integrated with other systems, modules, techniques, or methods without departing from the scope of the present disclosure. Other devices or components shown or discussed as coupled to, or in communication with, each other may be indirectly coupled through some intermediate device or component, whether electrically, mechanically, or otherwise. Other examples of changes, substitutions, and alterations are ascertainable by one skilled in the art and could be made without departing from the scope disclosed herein.

Many modifications and other embodiments of the present disclosure set forth herein will come to mind to one skilled in the art to which these embodiments pertain having the benefit of the teachings presented in the foregoing descriptions and the associated drawings. Therefore, it is to be understood that the disclosure are not to be limited to the specific embodiments disclosed and that modifications and other embodiments are intended to be included within the scope of the appended claims. Moreover, although the foregoing descriptions and the associated drawings describe example embodiments in the context of certain example combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. Although specific terms are employed herein, they are used in a generic and descriptive sense only and not for purposes of limitation.

1. An apparatus comprising:
a manifold defining a gas flow channel that is configured to convey a gaseous substance from a gas inlet on a surface of the manifold; and
at least one integrated flow regulating component comprising a filtering element that is configured to filter the gaseous substance, wherein the filtering element is disposed adjacent an orifice that leads to an interior channel of the integrated flow regulating component, and wherein the filtering element is disposed upstream with respect to the orifice.

2. The apparatus of claim 1, wherein filtering element is at least partially contained within a body of the integrated flow regulating component.

3. The apparatus of claim 1, wherein the filtering element is removably attached to at least a surface of the integrated flow regulating component.

4. The apparatus of claim 3, wherein the filtering element defines a cavity configured to abut the orifice.

5. The apparatus of claim 1, wherein the at least one integrated flow regulating component is configured to be removably received within an opening on a surface of the apparatus.

6. The apparatus of claim 5, wherein the apparatus comprises a gas detecting apparatus.

7. The apparatus of claim 1, wherein a diameter of the orifice is between 0.2 millimeters (mm) and 2 mm.

8. The apparatus of claim 1, wherein the filtering element comprises a substantially cylindrical member.

9. The apparatus of claim 1, wherein the filtering element comprises at least one of a textile or plastic material.

10. The apparatus of claim 1, wherein the at least one integrated flow regulating component comprises at least one of a metal or plastic material.

11. An integrated flow regulating component comprising:
a filtering element that is configured to filter a gaseous substance,
wherein the filtering element is disposed adjacent an orifice that leads to an interior channel of the integrated flow regulating component, and
wherein the filtering element is disposed upstream with respect to the orifice.

12. The integrated flow regulating component of claim 11, wherein filtering element is at least partially contained within a body of the integrated flow regulating component.

13. The integrated flow regulating component of claim 11, wherein the filtering element is removably attached to at least a surface of the integrated flow regulating component.

14. The integrated flow regulating component of claim 13, wherein the filtering element defines a cavity configured to abut the orifice.

15. The integrated flow regulating component of claim 11, wherein the integrated flow regulating component is configured to be removably received within an opening on a surface of an apparatus.

16. The integrated flow regulating component of claim 15, wherein the apparatus comprises a gas detecting apparatus.

17. The integrated flow regulating component of claim 11, wherein a diameter of the orifice is between 0.2 mm and 2 mm.

18. The integrated flow regulating component of claim 11, wherein the filtering element comprises a substantially cylindrical member.

19. The integrated flow regulating component of claim 11, wherein the filtering element comprises at least one of a textile or plastic material.

20. The integrated flow regulating component of claim 11, wherein the integrated flow regulating component comprises at least one of a metal or plastic material.
